(19) United States
(12) Patent Application Publication
(10) Pub. No.: US 2017/0304528 A1
(43) Pub. Date: Oct. 26, 2017

(54) IMPLANTABLE MEDICAL DEVICE FOR DELIVERING CELLS

(71) Applicant: DeepScience Ltd., Evergreen, CO (US)

(72) Inventor: Dahl Winters, Evergreen, CO (US)

(73) Assignee: DeepScience Ltd., Evergreen, CO (US)

(21) Appl. No.: 15/494,110

(22) Filed: Apr. 21, 2017

Related U.S. Application Data
(62) Division of application No. 62/326,517, filed on Apr. 22, 2016.

(51) Int. Cl. A61M 5/14; A61K 9/00; A61M 39/02; A61M 31/00

(52) U.S. Cl. CPC A61M 5/14; A61M 39/0208; A61K 9/0024; A61M 31/002

(57) ABSTRACT

An implantable and retrievable medical device is provided. The device may be implanted in and extracted from a patient and the device is adapted to house and deliver donor cells or other drugs. The device comprises a hollow core having a volume for receiving cells and a plurality of layers surrounding the core. The layers comprise various materials suitable for enhancing immunoprotection and for promoting vascular growth into the device.

This U.S. Non-Provisional Patent Application claims the benefit of priority from U.S. Provisional Application No. 62/326,517, filed Apr. 22, 2016, the entire disclosure of which is hereby incorporated by reference.

### FIELD

The present disclosure generally relates to implantable and retrievable medical devices. More specifically, embodiments of the present disclosure relate to devices that may be implanted in and extracted from a patient, and wherein the device is adapted to be accepted by a patient, promote vascularization and deliver donor cells to the patient.

### BACKGROUND

Implantation of donor cells or other foreign bodies into an affected patient have successfully accomplished treatment of various conditions and diseases. For example, pancreatic islet beta-cells are known to sense blood sugar levels and secrete insulin to maintain homeostasis. In patients with diabetes, however, islet beta-cells are either lacking or ineffective. Diabetes is a disease of the pancreatic islet cells wherein those affected lack adequate levels of insulin and have difficulty controlling their blood sugar. One alternative to self-administration of medicine and insulin is islet transplantation. The procedure involves an infusion of isolated donor islets into the patient. If the donor cells are accepted, these islets will function to regulate blood glucose levels through the production of insulin. Islet transplantation is therefore a treatment strategy that allows diabetics to reduce or eliminate the need for insulin injections to control their disease.

U.S. Pat. No. 5,984,890 to Gast et al., which is hereby incorporated by reference in its entirety, discloses a medical device for the implantation of solids within an animal. Gast et al. do not provide or describe a specific implant, but disclose various methods and devices for implanting devices as well as various needs and applications for doing so.

U.S. Pat. No. 5,391,164 to Giampapa, which is hereby incorporated by reference in its entirety, discloses an implantable multiple-agent biologic delivery system including a pod for subcutaneous implantation. Various features of Giampapa are contemplated for use in embodiments of the present disclosure. Giampapa fails to disclose, however, devices, methods and systems as described herein.

U.S. Pat. No. 5,484,403 to Yoakum et al., which is hereby incorporated by reference in its entirety, provides a hypodermic syringe for implanting solid objects. The devices and methods provided by Yoakum et al. are contemplated for use with embodiments of the present disclosure. Specifically, Yoakum et al. provides a device for injecting implantable solid objects which may be used to insert one or more implants of the present disclosure within an animal.

### SUMMARY

A long-felt and unmet need exists for a device and method that enables transplantation and subsequent retrieval or extraction of human islets and associated donor cells. Implantation and transplantation devices and methods of the present disclosure are not limited to those adapted for treating diabetes or any other specific condition. In various embodiments, devices and methods are described that are suitable for treatment of diabetes by islet transplantation. It will be expressly recognized, however, that the present disclosure is not limited to such methods, devices, or intended uses. Indeed, various applications and treatments are contemplated.

Although various embodiments contemplate the provision of donor islet cells and other cells within an implant and wherein the cells are ultimately provided to a patient, the present disclosure is not limited to implants comprising cells. It is contemplated that the devices and implants of the present disclosure may comprise various agents and materials including, but not limited to, cells, drugs, and various compounds that may be desirable to inject, insert or otherwise administer to a patient.

In various embodiments, an implant is provided, the implant generally comprising an islet transplantation device. The implant generally comprises a device for insertion within a patient, the device comprising a retrievable device adapted for treatment strategies including, but not limited to, islet transplantation.

In various embodiments, an implant comprising a hollow-core is provided. The implant comprises a shell enclosed in a soft alginate outer layer to provide immunoprotection and limit fibrosis. An outer coating of alginate and a vascularization inducer is provided to promote the growth of new blood vessels to the implant. In preferred embodiments, the implant comprises an islet transplantation device of generally cylindrical or pill-shaped construction that is approximately 2 mm in diameter and/or width and approximately 12 mm long. The implant is preferably rotationally symmetrical about a longitudinal axis, but may comprise other shapes. In alternative embodiments, the implant comprises a diameter or width of between approximately 0.50 mm and approximately 10 mm, and a length of between approximately 3 mm and approximately 50 mm. As one of skill in the art will recognize, the device is preferably sized so as to be accepted within an animal and such that it may be implanted and extracted using known technologies and devices. However, as the present disclosure provides devices, methods and systems that are not limited to specific animals (e.g. humans) or specific treatments, the device may comprise various dimensions.

Preferably, implants of the present disclosure are smaller than an ALZET™ osmotic pump and comprise a hollow core surrounded by at least two layers. In certain embodiments, a first layer comprises an alginate-polyacrylamide hybrid gel network, and a second layer comprises a soft, immunoprotective alginate outer layer. The first layer comprises a relatively stiff, tough hybrid gel to provide structural stability for subcutaneous implant insertion and removal. In various embodiments, devices and methods of the present disclosure provide implants that can be inserted into an intended location in approximately one to three minutes using standard procedures.

In various embodiments, a high level of islet function is promoted through at least one of several strategies. In certain embodiments, ultra-pure high-G alginate comprising the alginate-polyacrylamide matrix is covalently modified with ECM protein-specific peptides (RGD) to stimulate adhesion of islet cells. This alginate does not elicit an immune response. Vascularization is aided by recombinant VEGF-C that is distributed on the surface of the implant. As slow biodegradation of the outer alginate occurs, more VEGF-C is released, promoting extensive vascularization within a few days. During that time frame, oxygen is supplied through the gradual breakdown of calcium peroxide in the alginate-polyacrylamide matrix, as well as in the core matrix if oxygenation material is introduced by syringe along with the cells. After a certain period of time (e.g. 80 days), half the alginate mass of the implant may be lost, but structural stability is maintained by the alginate-polyacrylamide network, allowing the implant the associated cells or donor materials.

Alginates are polysaccharides that form an immunoisolating network, able to protect biofilm bacteria against phagocytosis in humans. Alginate is provided in various portions of implants of the present disclosure at least in part due to its immunoprotective properties.

In various embodiments, implants are provided with a hollow core adapted to be filled with cells suspended in partially polymerized high-G alginate and RGD for adhesion. An Alginate-Polyacrylamide (A-P) layer surrounds the hollow core, the A-P layer provided to increase stiffness, durability and oxygenation. An outer high-G alginate layer is provided around the A-P layer to enhance immunoprotection. An outermost layer is provided comprising high-G alginate and VEGF-C for vascularization induction and promotion of vascularization. In various embodiments, cells are inserted into the hollow core of the implant prior to completion of the outer high-G alginate layer and coating. Cell implantation is preferably accomplished by injection from a syringe needle or cannula.

Alginate use is known in tissue engineering, including clinical products using alginates and Phase II clinical trials involving alginate microcapsules supporting islet cells. The use of such alginates is contemplated in applications, methods and devices of the present disclosure.

Alginates with a G-content of 50% or above are recognized as not eliciting an immune response. In contrast, high-M alginates (70-80%) have been shown to stimulate immune cells in mice. This may be due to the presence of polycations in these studies involving high-M alginates, which by themselves stimulate the complement cascade and provoke an inflammatory reaction. It has been shown that beads made of different alginates, including high-M and high-G alginates with high molecular weight, performed similarly with a low degree of fibrosis when implanted subcutaneously in Wistar rats. High-M alginates may be preferred for implantation of pancreatic islets due to observed increased angiogenesis. However, this increased angiogenesis may be due to the smaller pore size of the high-M alginate creating an immune barrier to large molecules such as IgG (150 kDa), allowing more angiogenesis to proceed undisturbed. In various embodiments of the present disclosure, the use of high-G alginates is provided to reduce the immune response. High-G alginates have the additional advantage of not complexing as well with polycations compared to high-M alginates, reducing the chance of immune response by that route.

In various embodiments, a multilayer structure is provided to encapsulate the islet cells and offer greater structural strength to the implant as well as to isolate it from the immune system. Certain alginate encapsulation systems do not prevent protrusion of cells from the capsule, which leads to immunorejection, fibrosis, and eventually necrosis of the cells contained. An examination of the structural strength of a typical alginate gel offers insight into why protrusion is so common. The Young's modulus of an alginate gel can vary from 242+/−16 Pa to 1337+/−27 Pa. Soft tissues in general can range from a few kPa to a few hundred kPa, as exemplified by gelatin gels with similar Young's moduli. Thus, typical alginate gels have a stiffness no greater than that of the softest tissues. Rupture readily occurs when an alginate gel is stretched to about 1.2 times its original length, as might occur during implant extraction. However, a hybrid alginate-polyacrylamide gel strongly resists rupture, having a rigidity on the order of cartilage. The provision of such gels in implants of the present disclosure provides a device that is much easier to insert and remove. An extremely stretchable and tough hydrogel can be created by mixing two types of crosslinked polymer—ionically crosslinked alginate and covalently crosslinked polyacrylamide. The stress at rupture is known to be approximately 156 kPa for the hybrid gel, compared to only 3.7 kPa for the alginate gel alone and 11 kPa for the polyacrylamide gel alone. This is stiffer than soft tissue, but it is possible to make an even stiffer implant for sturdier implantation and extraction. Alginate-polyacrylamide hydrogels contemplated for use with embodiments of the present disclosure comprise both high stiffness and toughness, with elastic moduli on the order of 1 MPa.

According to the present invention, an effective administration protocol (i.e., administering a therapeutic composition in an effective manner) comprises suitable dose parameters and modes of administration that result in elicitation of an appropriate response in an animal that has a disease or condition, or that is at risk of contracting a disease or condition, preferably so that the animal is protected from the disease. A beneficial effect can easily be assessed by one of ordinary skill in the art and/or by a trained clinician who is treating the patient. Effective dose parameters can be determined using methods standard in the art for a particular disease. Such methods include, for example, determination of survival rates, side effects (i.e., toxicity) and progression or regression of disease.

In various embodiments, islets of the present disclosure comprise a diameter of between approximately 100-200 μm, and preferably of about 150 μm. The volume of 2000 islet equivalents (“IEQs”) is 3.53 cubic mm. The volume of a 2 mm×12 mm implant (of which approximately 25% of the total volume is available for cells) is approximately 9.42 cubic mm. The ratio of cell-containing volume to implant volume in certain embodiments of the present disclosure is thus approximately 0.375. Such embodiments provide room for both cell packing and subsequent growth. A person of ordinary skill in the art will appreciate that variations on the above concentrations, diameters, and volumes can be used to effectuate therapeutic dosages of islet cells in various animals. For example, the volume of 2,000 IEQs is effective in mice and 300,000-500,000 IEQs is effective in humans.

In certain embodiments, an outer alginate shell is provided with an implant. This shell is provided at least in part because alginate-polyacrylamide has been shown to create mild fibrosis compared to high-G alginate alone. As an example, it is known that a hybrid alginate-polyacrylamide gel was implanted in dorsal subcutaneous pockets in male Lewis rats for 8 weeks to assess inflammation, vascularization, and fibrosis. After eight weeks, the hybrid gel had been encapsulated with a fibrotic collagen encapsulation and showed new vasculature, but with the absence of macrophages or lymphocytic infiltrations that suggested a limited inflammatory response. Vascularization is desirable, but fibrosis should be kept to a minimum. Accordingly, embodiments of the present disclosure provide for an outer alginate shell and other features to limit fibrosis while still accomplishing objectives of the present disclosure.

Out of twenty five endogenous pro-angiogenic factors, vascular endothelial growth factor (VEGF) is the most studied regulator of vascular development. It shares 42% amino acid sequence identity with placental growth factor, and the placenta is an organ known for its rapid growth and vascularization. However, VEGF-C is a more potent promoter of angiogenesis than VEGF, and is thus contemplated for use as a means for promoting vascularization in implants of the present disclosure. To demonstrate the potency of VEGF-C, micropellets (0.35×0.35 mm) of sucrose aluminum sulfate have been known to be coated with hydron polymer type NCC to make them release their contents of either 160 ng of recombinant VEGF-C or VEGF slowly. These micropellets were implanted in the corneas of mice for five days and induced intensive neovascularization. Additionally, in vivo tests were performed on chicken embryos using methylcellulose disks containing 2.5 μg of VEGF-C or VEGF. The number of new vessel branches induced by VEGF-C in a 4-5 day incubation period was significantly greater than that induced by VEGF.

The MONOJECT™ AVID Injector is a syringe with a removable 12-gauge needle assembly. This device is known to be useful for injecting microchips into animals at both intramuscular as well as subcutaneous locations. In various embodiments, it is contemplated that this syringe, and/or various similar devices are useful for or provided as an implantation tool for implants of the present disclosure.

Cellular adhesion is important to cell survival, and how well cells adhere to and grow inside an implant depends on both the physical and chemical properties of the implant, particularly the surface of the implant. Islet cells in particular show greater survival when they are cultured in extracellular matrix proteins—fibronectin, collagen IV, or laminin. Collagen IV is an abundant material, but may not be suitable for use with certain embodiments of the present disclosure because it diminishes glucose-induced insulin responses. Alginate is therefore contemplated for use with embodiments of the disclosure. In certain embodiments, alginate is grafted with bioactive peptides such as the RGD sequence (Arg-Gly-Asp) found in ECM proteins, and cell adhesion is thereby promoted. Such embodiments preferably comprise a hollow core to facilitate insertion of a number of desired cell types. The cell suspension is perferrably mixed with partially polymerized alginate+RGD before introduction by syringe into the hollow core.

In various embodiments, a stiff alginate-polyacrylamide shell contains a chemical mechanism to diffuse oxygen to the cells in the hollow core. It is also contemplated that a partially polymerized alginate+RGD is mixed with the cells, which also contains the same oxygenation mechanism, thus allowing oxygen to be supplied directly next to the cells and ensuring a higher concentration than from the alginate-polyacrylamide shell alone. The oxygenation mechanism involves the following reaction:

2CaO₂+2H₂O→O₂+2Ca(OH)₂

To mitigate the expected production of H₂O₂ from a competing reaction that takes place at physiological pH, catalase, which generates O₂ from H₂O₂, accompanies the oxygenation mechanism in both the alginate-polyacrylamide shell as well as when added with cells in the hollow core. CaO₂ maintains its oxygen-releasing capacity over a period of days to weeks due to its low solubility, but generation of insoluble products of CaO₂ and increasing alkalinity of the surrounding solution have been problematic for cell survival. To neutralize the resulting alkalinity, the alginate is crosslinked with Al³⁺, the most optimal known crosslinking ion in terms of allowing high oxygen-releasing efficiency, slow release kinetics, and good pH buffer capacity. The H₃O+ generation through the hydrolysis of the released trivalent cations effectively neutralizes the pH increase caused by the oxygen-releasing process, yielding a neutral species (a hydrated metal hydroxide). The target quantity of CaO₂ necessary to ensure adequate initial oxygenation (up to 1 week) for islet cells is estimated at 5% (w/v) with the following rationale. It has been shown that rapid decomposition of a 0.2% (w/v) slurry of CaO₂ at pH 7; at this pH, H₂O₂ was produced more rapidly and at higher quantities than 02. A 2% (w/v) mixture has been shown to yield adequate oxygenation duration within alginate beads, at least in the absence of cells. For example, up to 10 days of O₂ release has been demonstrated from 1, 5, and 10% concentrations of CaO₂ for 3T3 fibroblasts. The greatest cell growth occurred with the 5 wt % concentration.

VEGF-C is a more potent promoter of angiogenesis than VEGF, which is the most studied regulator of vascular development. Various embodiments of the present disclosure therefore provide VEGF-C as a means for promoting vascularization to an implant. A coating of VEGF-C is applied to the outside of the soft alginate outer layer, promoting vascularization on the surface of the implant. However, despite the external coating, it is possible due to the softness of this outer alginate layer that capillaries may grow into the implant. This breach of the outermost immunoprotective layer may result in some fibrosis due to contact with the polyacrylamide component of the alginate-polyacrylamide layer. However, the pore size of the alginate-polyacrylamide layer is such that antibodies cannot penetrate the layer, and its stiffness makes it unlikely that capillaries will quickly breach this layer. This should hold true until significant biodegradation occurs.

In one embodiment, an implantable cell delivery device is provided. The device is adapted to be inserted into the tissue of an animal and comprises a shell comprising a core operable to receive and store cells and a first layer provided to enhance immunoprotection and limit fibrosis. A second layer is provided, the second layer comprising an alginate polyacrylamide layer and having a stiffness. A third layer is provided comprising a vascularization inducer to promote the growth of new blood vessels to the device.

In further embodiments, implants are provided in a device as small as 4×4×1.5 cm or larger that can be implanted under the skin of an animal. The device as small as 4×4×1.5 cm can be comprised of up to 150 or fewer of the inner two layers of the implant design as disclosed herein and arranged in flat bundles of six implants. In an embodiment of a device larger than 4×4×1.5 cm, more than 150 of the inner two layers of the implants may be provided.

In further embodiments, the flat bundles of six implants can be layered in a device with or without the provision of an inner gap within the layered bundles of implants.

The Summary is neither intended nor should it be construed as being representative of the full extent and scope of the present disclosure. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the drawings.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the embodiments.

FIG. 1 is a perspective view of an implant according to one embodiment of the present disclosure, with various features shown in phantom for illustrative purposes.

FIG. 2 is an illustration of a method of implanting an implant according to various embodiments of the present disclosure.

FIG. 3 is a side view of a syringe for implanting solid objects according to various embodiments of the present disclosure.

FIGS. 4A-4C are views of designs for implantation of multiple implants according to embodiments of the present disclosure.

FIG. 4A is a plan view of an arrangement of multiple implants according to one embodiment of the present disclosure.

FIG. 4B is a detailed perspective view of an implant for use with the embodiment of FIG. 4A.

FIG. 4C is a plan view of an arrangement of multiple implants according to one embodiment of the present disclosure.

FIG. 4D is a detailed perspective view of an implant for use with the embodiment of FIG. 4C.

FIG. 4E is an exploded perspective view of the arrangement of the embodiment of FIG. 4C.

FIG. 4F is a plan view of an arrangement of multiple implants according to one embodiment of the present disclosure.

FIG. 4G is a detailed perspective view of an implant for use with the embodiment of FIG. 4F.

FIG. 4H is an exploded perspective view of the arrangement of the embodiment of FIG. 4F.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

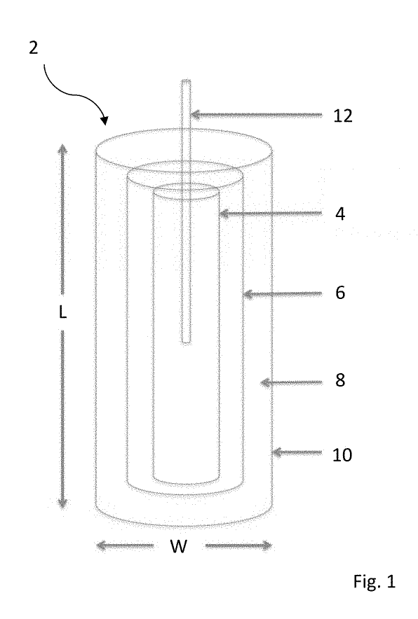

FIG. 1 is a perspective view of an implant according to one embodiment of the present disclosure, with various features shown in phantom for illustrative purposes. As shown in FIG. 1, the implant 2 comprises a substantially cylindrical shape. The embodiment of FIG. 1 provides a device with an outer surface or shape that comprises a rotationally symmetrical cylinder. It will be recognized, however, that implants 2 of the present disclosure may comprise various shapes, including pill shapes (i.e. cylinders with rounded ends), ovoid shapes, circular shapes, rectilinear shapes, etc. Accordingly, no limitation is provided herewith respect to the outer shape and dimensions of the insert(s). Preferably, the insert 2 comprises outer dimensions including a length L and a width W. In certain embodiments, the length L comprises a distance of between approximately 10.0 millimeters and 15.0 millimeters, and preferably of approximately 12.0 millimeters. In certain embodiments, the width W or diameter of the insert 2 comprises a distance of between approximately 1.0 and 5.0 millimeters, and preferably of approximately 2.0 millimeters. Thus, in preferred embodiments, inserts are provided comprising a length L of approximately 12.0 millimeters and a width W of approximately 2.0 mm. Although various alternative sizes and proportions are contemplated, inserts of preferred embodiments of the disclosure have been determined to provide a suitable interior volume while also being of the appropriate size and dimensions to be accommodated by various insertion and extraction devices.

The implant 2 of the embodiment of FIG. 1 comprises a hollow core 4. The hollow core 4 is operable to be filled with donor cells that are ultimately to be implanted into a patient. In preferred embodiments, the cells are suspended in partially polymerized high-G alginate and RGD for adhesion. The hollow core 4 is surrounded by a first layer 6, wherein the first layer 6 comprises alginate-polyacrylamide for stiffness, durability, and to promote oxygenation. The first layer 6 is further surrounded by a second layer 8, wherein the second layer 8 comprises a high-G alginate layer for immunoprotection. Additionally, and as shown in the embodiment of FIG. 1, the insert 2 comprises a third layer 10. The third layer 10 comprises a coating of high-G alginate and VEGF-C for vascularization induction and the promotion of a host vascular system receiving and accepting the implant.

As shown in FIG. 1, an implant 2 is provided comprising a multi-layer construction. The multilayer implant 2 provides greater structural strength to the implant, and provides isolation of at least certain portions of the implant from the immune system. The third layer 10 comprises an alginate shell to reduce the risk of fibrosis, while also promoting vascularization and vascular growth from the host into the implant 2 to facilitate acceptance of donor cells.

A syringe tip 12 is provided to insert cells into the hollow core 4. Insertion of cells via the syringe 12 occurs at least prior to completion and formation of the third layer 10, and preferably occurs prior to completion of the second and third layers 8, 10.

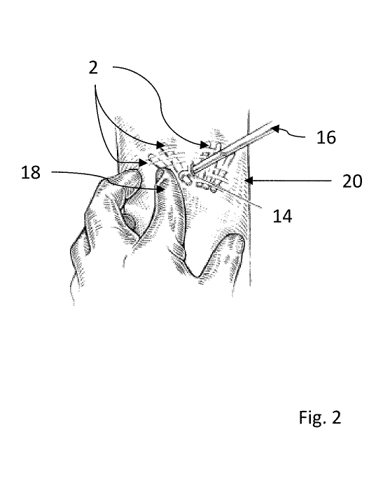

FIG. 2 is a perspective view of a method of removal of implants 2 according to various embodiments of the present disclosure. As shown, a plurality of implants 2 is provided within a patient 20. Although five separate implants 2 are provided within the patent 20 in FIG. 2, it will be recognized that removal techniques as shown and described herein may be performed with as few as one implant. The implants 2 are provided subcutaneously in the patient 20, but may be provided as implants in various regions or portions of a patient's anatomy. It is contemplated that implants 2 of the present disclosure may be removed from a patient in approximately one to three minutes. One method of implant removal contemplated by the present disclosure comprises cleansing and/or disinfecting the incision site 14, administering subcutaneous anesthesia, and making an incision that is preferably parallel to the longitudinal axis of an implant 2. The implant is then palpated by a finger 18, and at least a portion of the implant is forced through the incision. A tool 16 (e.g. forceps) is then used to grasp and extract the implant(s) 2. It is contemplated that due to the use and presence of VEGF-C in implants 2 of the present disclosure, extensive vascularization will be present in and around the implantation site. Accordingly, removal methods in accordance with embodiments of the present disclosure contemplate a further step of cutting and/or removing this vasculature before or after implant removal. An anesthetic with epinephrine is preferably provided to promote vasoconstriction and thus reduce bleeding during this method.

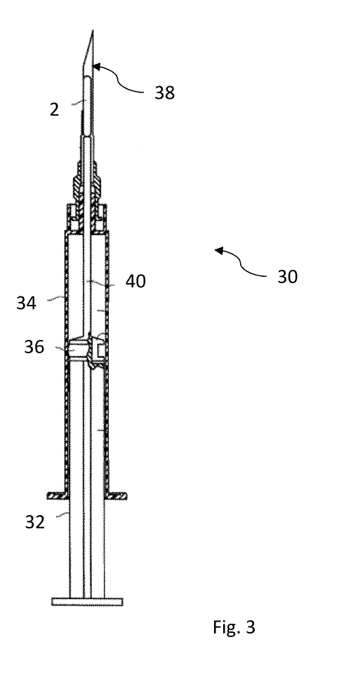

FIG. 3 is a side view of a syringe for implanting solid objects according to various embodiments of the present disclosure. As shown, the syringe 30 comprises a plunger rod 32 within a barrel 34. A synthetic rubber gasket 36 provides a user with the feel of a conventional fluid-injecting hypodermic syringe. The purpose of the gasket 36 is to provide a frictional force that resists the movement of the plunger 32. Since there is no need for a leak-proof seal for a solid-object-implanting syringe, the gasket can 36 be made of a porous material or air channels can be incorporated in the gasket 36 to allow air to pass freely through the gasket, thereby avoiding air pressure build-up in the barrel that might force air through the cannula 38 and the incision in the body during the implantation procedure. An implant 2 in accordance with embodiments of the present disclosure is provided in the cannula 38 and is ready for implantation in the illustration of FIG. 3. Application of force to the plunger rod 32 displaces a push rod 40 which forces the implant 2 out of the syringe 30.

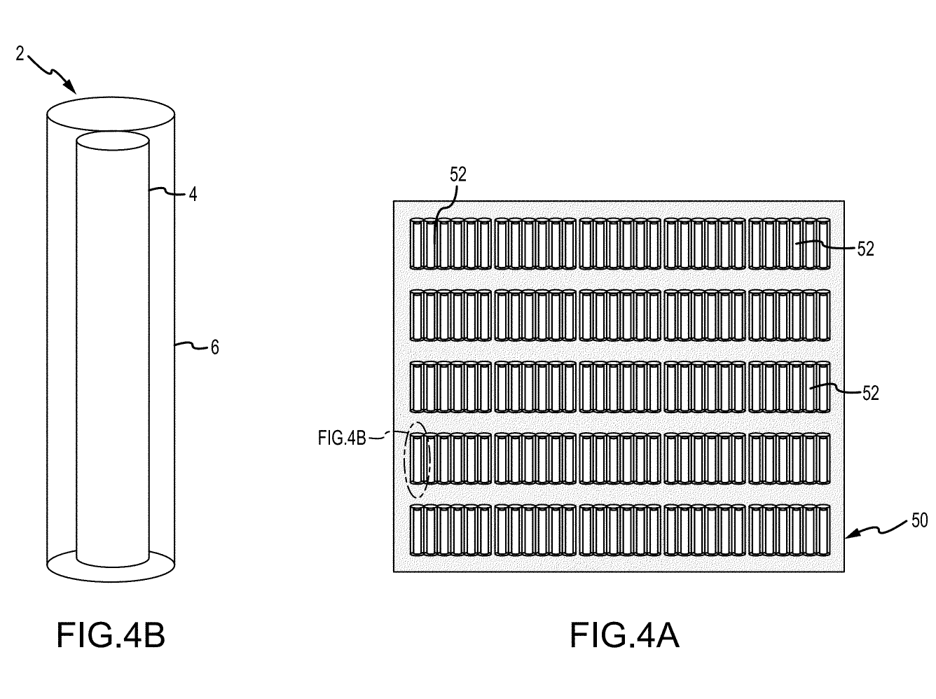

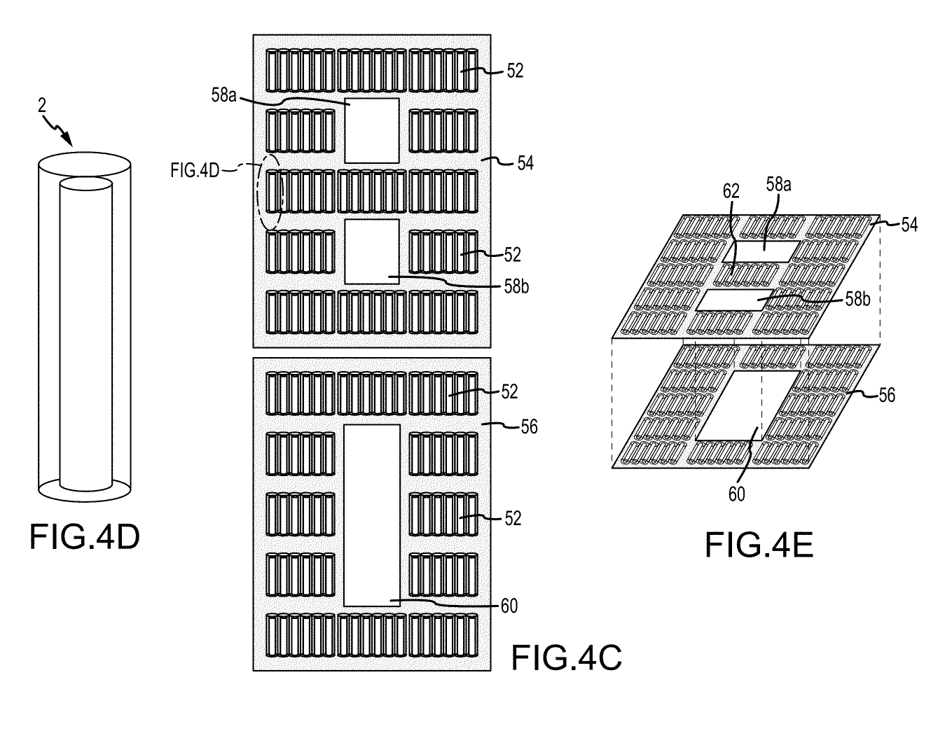

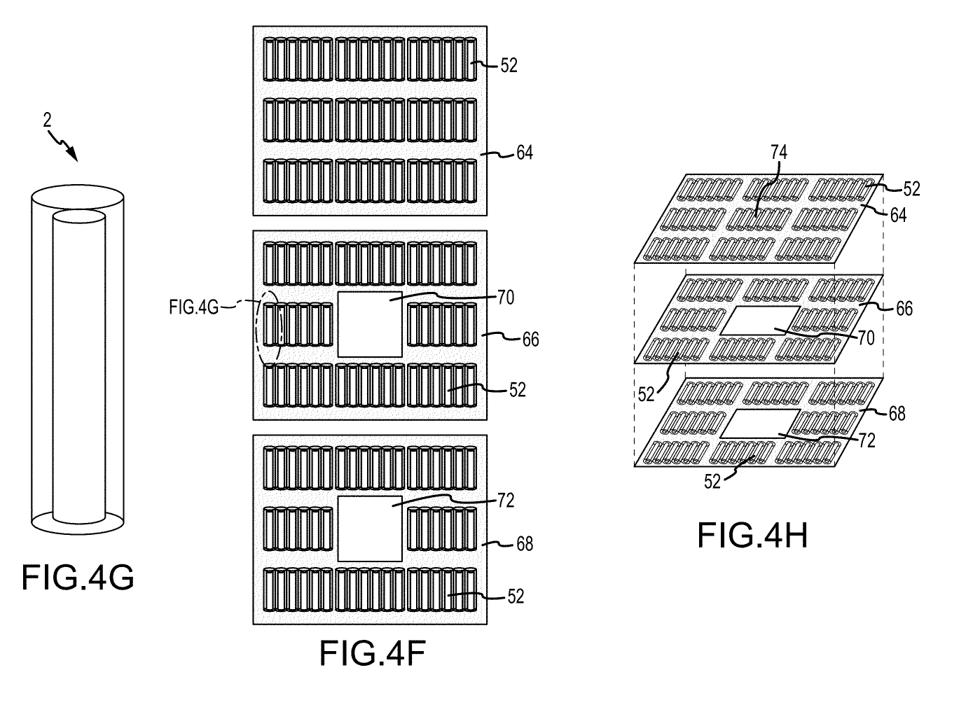

FIGS. 4A-4C are views of designs for implantation of multiple implants according to embodiments of the present disclosure. In one embodiment, and as shown in FIG. 4A, a plurality of implants 2 are provided on a sheet 50. The sheet 50 comprises twenty-five flat bundles 52 of implants 2 disposed on a flat surface. In the depicted embodiment, the flat surface of the sheet 50 comprises alginate-polyacrylamide providing structural stiffness to hold the implants 2 within the sheet 50. In the depicted embodiment, each bundle 52 comprises six implants. Thus, as depicted in FIGS. 4A-4B, the device comprises 150 of the inner two layers 4, 6 of the implant design as disclosed herein and arranged in flat orientation. These flat bundles of implants 52 are contemplated as being bonded together with alginate-polyacrylamide (for example) for adequate structural stiffness for removing the implant as a whole, while allowing enough flexibility to rest under the skin.

In further embodiments, and as shown and described herein, multiple layers of flat bundles of implants can be provided. The device may comprise a single layer, two layers, three layers, or more. FIG. 4A depicts the flat bundles of implants 52 oriented in a single layer 50.

FIG. 4C depicts a plurality of implants provided in a two-layer arrangement, wherein a first sheet 54 and a second sheet 56 are provided. Each of the sheets 54, 56 comprises a plurality of bundles 52 of implants 2, an example of which is shown in the detailed perspective view of FIG. 4D. The first sheet 54 comprises a plurality of spaced-apart gaps or voids 58a, 58b. The second sheet 56 comprises a single void 60 that is operable to and intended to at least partially align with the voids 58a, 58b of the first sheet when the first sheet 54 and the second sheet 56 are stacked or aligned. The voids 58a, 58b, 60 comprise apertures that are devoid of material and allow for transmission of materials including, for example, vasculature and tissue that is to grow in and around the device subsequent to implantation.

FIG. 4E is a perspective view of the first and second sheets 54, 56, which are intended to be stacked or layered. As shown, the voids 58a, 58b, 60 are positioned such that they at least partially align upon layering the sheets. The larger aperture 60 of the second sheet 56 provides for at least one bundle 62 to be exposed on both sides of the bundle, and the voids generally serve to allow for in-growth of tissue and vasculature subsequent to implantation of the sheet(s). In various embodiments, stacked or layered sheets comprise an alginate-based gel outer coating including vascular endothelial growth factor C (VEGF-C). This outer coating may be used to limit fibrosis and stimulate vascularization.

FIG. 4F is a top plan view of a plurality of implants provided on sheets. Specifically, a first 64, second 66 and third sheet 68 are provided. Each of the sheets 64, 66, 68 are provided with a plurality of bundles of implants 52, and the sheets are operable to be stacked or layered. At least some of the sheets comprise apertures or void spaces. Specifically, and as shown in the embodiment of FIG. 4F, the second and third sheets 66, 68 comprise first and second apertures 70, 72. The apertures generally comprise areas that are devoid of material and allow for transmission of fluids and tissue. FIG. 4G is a detailed view of an implant 2 that is provided within a bundle 52. The bundles of the depicted embodiment comprise six implants, which comprise implant structure(s) as shown and described herein.

FIG. 4H is an exploded perspective view of the first, second and third sheets 64, 66, 68 of FIG. 4F. The sheets comprise the same or similar length and width dimensions and are operable to be layered or stacked. The apertures 70, 72 provided in the second and third sheets 66, 68 provide that at least some of the implants of the first sheet 64 are exposed on both sides, even when the sheets are stacked in a three-layer orientation. Although FIG. 4H provides the first, second and third layers in a specific orientation, alternative embodiments are contemplated. For example, the second layer 66 and the first layer 64 may be transposed, such that layers with apertures are provided on the top and bottom and a middle layer is devoid of an aperture. Similar to the embodiment shown in FIG. 4E, this embodiment of stacked or layered first, second and third sheets 64, 66, 68 comprises an alginate-based gel outer coating including vascular endothelial growth factor C (VEGF-C). This outer coating may be used to limit fibrosis and stimulate vascularization.

In the instance of multiple layers of implant bundles, a gap or void is provided to allow for ingrowth of vasculature. Further, the inner gap can be surrounded as a group by the third layer which is the soft alginate shell containing VEGF-C. Such embodiments provide for a retrievable implant made possible, for example, through a 2 cm incision. In these embodiments of layered implant bundles, the implant bundles in layers are implanted under the animal's skin and subsequently unrolled so that the top layer of implant bundles rests flat under the skin. Removal of the implants can be done through an outpatient procedure.

In various embodiments, sheets or layers or implants are provided for insertion. No limitation with respect to the number of implants to be inserted within a patient is provided herein. However, in some embodiments, methods and devices are contemplated wherein between approximately 40 and approximately 200 implants as shown and described herein are provided for implantation within a patient.

While various embodiments of the present disclosure have been described in detail, it is apparent that modifications and alterations of those embodiments will occur to those skilled in the art. It is to be expressly understood that such modifications and alterations are within the scope and spirit of the present disclosure. Further, it is to be understood that the phraseology and terminology used herein is for the purposes of description and should not be regarded as limiting. The use of “including,” “comprising,” or “having” and variations thereof herein are meant to encompass the items listed thereafter and equivalents thereof, as well as, additional items.

What is claimed is:

1. An implantable cell delivery device adapted to be inserted into the tissue of an animal, the device comprising:
a shell comprising a core operable to receive and store cells and a first layer provided to enhance immunoprotection and limit fibrosis;
a second layer comprising an alginate polyacrylamide layer and having a stiffness; and
a third layer comprising a vascularization inducer to promote the growth of new blood vessels to the device.

2. The implantable cell delivery device of claim 1, wherein the vascularization inducer comprises a high-G alginate and VEGF-C.

3. The implantable cell delivery device of claim 1, wherein the device comprises a diameter of approximately 2.0 mm.

4. The implantable cell delivery device of claim 1, wherein the device comprises a length of approximately 12.0 mm.

5. The implantable cell delivery device of claim 1, wherein the core comprises donor cells.

6. The implantable cell delivery device of claim 1, further comprising a plurality of additional cell delivery devices of the same construction and wherein the cell delivery devices are bonded together.

7. The implantable cell delivery device of claim 1, wherein the cell delivery devices are bonded together with alginate-polyacrylamide.

8. An implantable cell delivery device adapted to be inserted into the tissue of an animal, the device comprising:
a sheet comprising a plurality of implants bonded together with alginate-polyacrylamide;
wherein each of the plurality of implants comprises a core operable to receive and store cells and a first layer provided to enhance immunoprotection and limit fibrosis, a second layer comprising an alginate polyacrylamide layer and having a stiffness; and a third layer comprising a vascularization inducer; and
wherein the sheet is operable to be rolled or folded for implantation within an animal.

9. The implantable cell delivery device of claim 8, wherein the vascularization inducer comprises a high-G alginate and VEGF-C.

10. The implantable cell delivery device of claim 8, wherein each of the plurality of implants comprises a diameter of approximately 2.0 mm.

11. The implantable cell delivery device of claim 8, wherein each of the plurality of implants comprises a length of approximately 12.0 mm.

12. The implantable cell delivery device of claim 8, wherein the core of each of the plurality of implants comprises donor cells.

13. The implantable cell delivery device of claim 8, wherein the sheet comprises alginate-polyacrylamide.

14. The implantable cell delivery device of claim 8, wherein at least some of the implants are bonded together with alginate-polyacrylamide.

15. A method of implanting a cell delivery device in an animal, the method comprising:
providing an implantable cell delivery device comprising: a shell comprising a core operable to receive and store cells and a first layer provided to enhance immunoprotection and limit fibrosis; a second layer comprising an alginate polyacrylamide layer; and a third layer operable to promote the growth of new blood vessels to the cell delivery device;
forming an incision in the dermis of an animal;
implanting the cell delivery device within the incision and such that the cell delivery device is provided subcutaneously in the animal;
closing the incision and allowing the cell delivery device to remain in the animal, wherein the cell delivery device is operable to promote vascularization induction between the animal and at least one of the first layer, the second layer, and the third layer.

16. The method of claim 15, wherein the animal is a human.

17. The method of claim 15, wherein a plurality of implantable cell delivery devices are provided.

18. The method of claim 17, wherein the plurality of implantable cell delivery devices are provided on a sheet, and further comprising a step of unrolling, unfolding, or spreading the sheet subsequent to inserting the sheet within the incision.

19. The method of claim 15, wherein the third layer comprises a vascularization inducer.

20. The method of claim 15, further comprising the step of removing the cell delivery device from the animal after a predetermined amount of time has elapsed.
